(19) United States
(12) Patent Application Publication
(10) Pub. No.: US 2006/0030921 A1
(43) Pub. Date: Feb. 9, 2006

(54) Intravascular securement device

(75) Inventor: Jack Chu, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/911,042

(22) Filed: Aug. 3, 2004

(51) Int. Cl. A61F 2/06

(52) U.S. Cl. 623/1.11

(57) ABSTRACT

A method and apparatus for a securement device useful for the treatment of aneurysms includes a hub and, in one aspect, a plurality of arms or spikes in a star pattern extendable therefrom and into engagement with a blood vessel wall. The securement device may be deployed to anchor a secondary device, such as an exclusion device for example a stent graft, in position in a flow lumen and thereby prevent the migration of the exclusion device in the flow lumen. The arms may be positioned to penetrate through the exclusion device and thence into the flow lumen wall to provide such securement.

### BACKGROUND OF THE INVENTION

1. Field of the Invention

Embodiments of the present invention generally relate to the field of the treatment of body lumens, more particularly to the field of the treatment of blood vessels, and more particularly to the treatment of blood vessel aneurysms with intraluminal devices such as stents, lined stents such as stent grafts, and the use of a securement device therewith to secure the intraluminal device in an intended position within the blood vessel.

2. Description of the Related Art

Aneurysm, i.e., the enlargement of a blood vessel at a specific location therein to the point where rupture of the blood vessel is imminent, has been treated in the past by surgical intervention techniques, whereby the affected portion of the blood vessel is removed, or bypassed, so that a synthetic graft replaces the flow lumen. This treatment regimen is highly invasive for the patient undergoing it, and typically requires a multiple day post-operative hospital stay, as well as several months of recovery time until the patient has fully recovered from the surgery. Additionally, some patients may not be capable of undergoing such a procedure.

To address the limitations imposed by surgical intervention to replace the aneurysmal blood vessel region with an artificial graft, a technique has been developed by which the aneurysmal blood vessel site is treated by placing what is known in the art as a stent graft, within the blood vessel in a position by which the tubular body of the stent graft spans the interior of the weakened area of the blood vessel wall. The stent graft, properly positioned, allows blood to flow through the hollow tubular interior, thereof, and also prevents blood, under systemic pressure, from reaching the weakened blood vessel wall at the aneurysmal site. The stent graft includes a graft portion provided to channel blood passage therethrough, and a stent portion, which supports the graft portion to maintain it in a hollow tubular configuration and press the graft portion against the blood vessel wall at locations remote from the aneurysmal site to seal off the aneurysm from further blood flow. Despite the intended expansion and sealing capability of the stent graft to the blood vessel wall, it is still possible, on occasion, for the stent graft to become dislodged or improperly sealed against the blood vessel wall. In such a case, fresh blood will reach the weakened aneurysmal wall location, creating a renewed risk that the blood vessel may rupture. Furthermore, if the stent graft is inadequately engaged against the blood vessel wall, such that the seal with healthy tissue is lost, it may migrate. In a graft having fenestrations, the wall of the stent graft around the fenestration could slip to become positioned over (obstruct) a branch artery, such as the renal arteries inducing renal blood starvation and potential renal failure.

It is known, in the art, to provide anchoring of the stent graft to the blood vessel wall by the provision of individual hooks connected to stents at multiple discrete locations about the circumference of the stent graft. These individual securement devices are deployed using a catheter or tube protecting the hook ends. The end of the catheter is tracked through a blood vessel to the securement location and when in position the hooks are extended through the wall of the stent graft and into the wall of the blood vessel. This process is both cumbersome and time consuming. In particular, the known securement devices which provide individual hooks are manipulated into place and located against the stent graft to pierce the stent graft and then enter the blood vessel wall. Deployment hooks or hooks attached to stents can require intricate twisting and pulling motions by the surgeon on the end of a wire or tube external to the body. This complex procedure is typically repeated to provide stents with hooks or hooks alone at several locations about the inner circumference or near the ends of the stent graft. Additionally, the placement of such hooks can distort the stent graft, as hook placement through the stent graft may be angled and therefore not, circumferentially, match the subsequent placement of the hook in the blood vessel wall. This can create a distortion about the circumference of the stent graft, thereby reducing the sealing capability of the stent graft allowing the leakage of blood into the aneurysmal location. Additionally, such hooks are used to anchor other intraluminal devices, such as those intended for in situ therapeutic materials delivery to the blood vessel site.

Therefore, there exists a need in the art for a stent graft securement system, which is easily deployable and results in securement of the stent graft to the blood vessel wall without circumferential distortion, with rapid deployment capability and verifiable positioning.

### SUMMARY OF THE INVENTION

The present invention generally concerns methods and apparatus for the placement and securement of intraluminal devices, such as a stent graft, in a body flow lumen location with minimal risk of endoleak and minimal risk of migration of the device from the intended location to an additional location in the lumen. In one embodiment, the invention includes an intraluminal exclusion device (exclusion device), such as a stent graft, having an outer cylinder-like wall which is located to span the location of a luminal risk factor, such as an aneurysm, and a securement device deployable to extend through the stent graft wall and into the blood vessel wall, to secure the stent graft to the blood vessel wall. The securement device may be placed in the body flow lumen at the time of intraluminal exclusion device placement or at some time thereafter where risk of intraluminal exclusion device migration becomes apparent. Additionally, the securement device may be used in conjunction with a device or material other than an intraluminal exclusion device, such as a device intended for the time release of therapeutic agents to the aneurysmal site, or may include such a delivery device formed therewith.

In a further embodiment, the securement device is provided, and includes at least one anchor portion extending from a main body portion. The anchor portion extends through the intraluminal exclusion device wall and into the flow lumen wall there adjacent. The securement device, in one embodiment, includes at least three anchor portions extending from a main body portion, where at least one of the anchor portions has a tip that is configured to pierce the intraluminal exclusion device and flow lumen wall with a limiter extending therefrom that is configured to bear against the inner surface of the intraluminal exclusion device and thereby limiting the penetration depth of the anchor into the blood vessel wall. In a still further embodiment, the securement device deploys without the need for extensive manipulation of a catheter or tube. Instead, upon release from the tube or catheter at the deployment location, the anchor portion of the securement device is deployed adjacent to its final anchoring position, i.e., adjacent the blood vessel wall, simply upon release thereof from the catheter or tube. Alternatively, the anchor portion of the securement device may be deployed through the simple inflation of a balloon. Thus, the securement device may be deployed without the need for excessive twisting and manipulation of a catheter or tube from a position remote from the securement device deployment position. Once the securement device is deployed and position, in one embodiment, it is anchored into the blood vessel wall by the simple act of pulling on a wire detachably connected therewith.

In a method of deploying the anchoring device, a catheter or tube capable of intravenous deployment is provided, and the securement device is located therein for deployment through a flow lumen, such as a leg artery, and thence to the intended position of securement of the intraluminal exclusion device. The tube includes an inner push rod capable of holding the securement device and moving it with respect to the flow lumen, as well as being releasable from the securement device once positioned in the flow lumen. Once the tip of the catheter or tube is positioned adjacent a deployment location, the securement device is deployed therefrom by maintaining the push rod stationary while the tube is withdrawn slightly from the deployment location, thereby pulling the tube past the securement device thereby locating the securement device in position in the flow lumen. The push rod is then pulled, to engage the tip of the securement device into the body flow lumen, after first piercing the exclusion device where the securement device is deployed in conjunction with an exclusion device. In one embodiment, the push rod includes one or more fluid passages therethrough, and a first balloon is positioned thereon at a location where the push rod extends through the securement device, and such balloon is inflatable to hold the securement device on the push rod while the securement device is still positioned within the tube or catheter. The tube or catheter is then further withdrawn, while holding the push rod stationary, to deploy the securement device out the end of the tube, whereby the anchor portions of the securement device are then extended into a position such that the tips thereof are engaged against the inner wall of the exclusion device. In one aspect, this is provided by configuring the securement device of a shape memory material, such that when the securement device is released from the tube, the anchor portions expand of their own accord into a shape to position the tips against the interior wall portion of the exclusion device about the inner circumference thereof. Alternatively, a second balloon may be provided on the push rod adjacent the anchor portions to urge the anchor portions outwardly to engage against the inner wall portion of the exclusion device. Thence, once the anchor portions are positioned with the tips against the inner wall of the exclusion device, the push rod extending through the securement device is retracted with respect to the tube or body, causing the first and second balloons to engage against the securement device and thereby move the securement device to cause the tips to pierce the wall of the exclusion device and the blood vessel wall, thereby securing the exclusion device in position. The first balloon is then deflated, (as is the second balloon, where used) thereby allowing the push rod, with the deflated balloon thereon, to be withdrawn through the securement device, leaving the securement device in position in the blood vessel wall. The catheter or tube, along with the push rod, is then retracted from the leg artery and the artery and entry cut in the leg are sutured shut.

The placement of the securement device may occur simultaneously with, or immediately after, placement of the exclusion device, or at a later time where risk of migration of the exclusion device is indicated. Additionally, the securement device may be deployed independently of an exclusion device, such that the securement device may secure an additional or different intraluminal device therein, or, may include such a device as an integral part thereof. In such a case, the tips of the securement device will, in one embodiment, directly pierce the blood vessel wall without first passing through an intermediate member such as an exclusion device.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings.

FIG. 1 is a schematic cross sectional view of a aneurysmal human ascending aorta, showing an exclusion device, in this embodiment, a stent graft, deployed therein;

FIG. 2 is a schematic cross sectional view, showing an aneurysmal thoracic aorta and an exclusion device, specifically a stent graft, deployed across the aneurysmal portion of the aorta;

FIG. 3 is a further sectional view of the aneurysmal aorta and stent graft of FIG. 1, having a securement device deployed therein;

FIG. 3a is a top, plan view of the securement device deployed in FIG. 3;

FIG. 3b is a sectional view of the securement device of FIG. 3a taken at 3b-3b;

FIG. 4 is a plan view of the preform of the attachment device of the present invention;

FIG. 5 is a side view of the preform of the attachment device of FIG. 4 having been cooled in a cooling media and bent to be positioned within an intraluminal delivery tube;

FIG. 6 is a partial view, partially cutaway, of a delivery tube and securement device ready to deploy the securement device in a flow lumen;

FIG. 7 is a view, in partial cutaway, of the lower aorta having a stent graft deployed therein, with the end of the tube 70 of FIG. 6 disposed adjacent the blood flow entry end of the stent graft for deployment of the securement device;

FIG. 8 is a further view of the stent graft and aorta of FIG. 7, showing the securement device being deployed;

FIG. 9 is a further view of the stent graft and aorta of FIG. 8, showing the securement device deployed from the tube but not yet secured in place;

FIG. 10 is a further view of the stent graft and aorta of FIG. 9, showing the securement device deployed in the aorta and securing the stent graft to the aorta wall; and

FIG. 11 is a further view of the stent graft and aorta, with the stent graft shown in partial cutaway, showing the tube being retracted from the deployment position.

### DETAILED DESCRIPTION

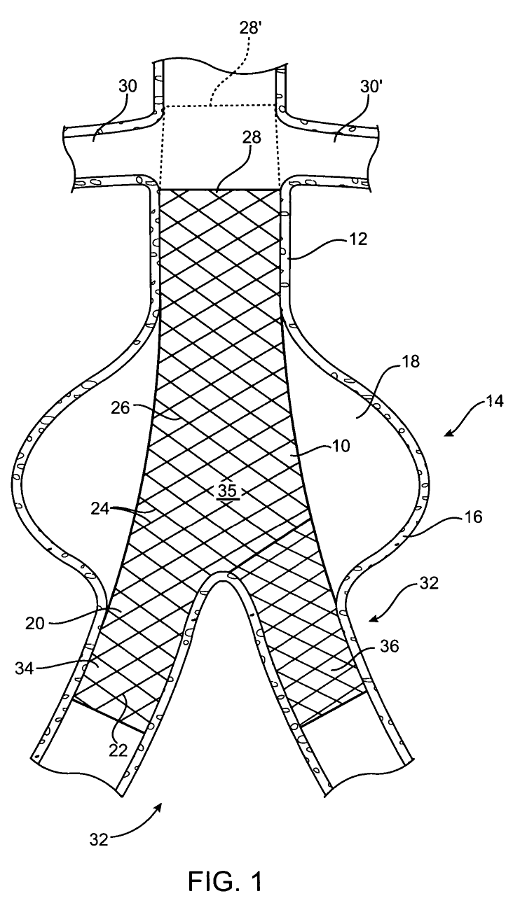

Referring initially to FIG. 1, there is shown an intravascular repair vehicle, specifically a stent graft 10, positioned in a blood vessel, in this embodiment, the descending abdominal aorta 12, and spanning, within the aorta 12, an aneurysmal portion 14 of the aorta 12. The aneurysmal portion 14 is formed of a bulging of the aorta wall 16, in a location where the strength and resiliency of the aorta wall 16 is weakened. As a result, an aneurysmal sac 18 is formed of distended vessel wall tissue. The stent graft 10 is positioned spanning the sac 18 and thereby both provides a secure passageway for blood flow through the aorta 12 and seals off the aneurysmal portion 14 of the aorta 12 from contact with further blood flow through the aorta 12. The stent graft 10 further includes a graft portion 20, which is configured from a biocompatible fabric, and which is sewn or otherwise attached to stent portion 22, which is shown as a plurality of wires 24 interleaved into a mesh 26 pattern (though any number of stent graft structures are well known in the art). The wires are preferably made from a shape memory material, such as nitinol, which may be cooled, in the desired shape, in liquid nitrogen or otherwise and when cold compressed or rolled into a shape which will fit within a delivery tube such as a catheter, and inserted therein cold. Once deployed from the catheter at room or body temperature, the wires regain the shape they had when originally cooled. The upper, or blood entry end 28 of the stent graft is positioned such that the renal arteries 30, 30′ are not occluded, i.e., below or downstream thereof. The lower or blood exit end 32 of the stent graft 10 is bifurcated into two branches 34, 36, each branch deployed to extend into and secure against the iliac branch arteries extending downstream from the aneurysm. The main body portion 35 of the stent graft 10 forms the upper end thereof, while branch 34 is preferably integrally formed with body portion 35, and branch 36 is provided as a separate element which is combined, in situ in the patient, to form the bifurcated stent graft.

Blood flowing through the stent graft 10 is not continuous in pressure or flow, and in fact the pressure can fluctuate substantially, causing expansion and contraction of the stent graft, as well as axial, i.e., along the flow direction of the blood, forces on the stent graft 10. It has been found that these forces can be sufficient to disengage the stent graft ends from the blood vessel wall, such that the stent graft 10 can migrate upwardly (against blood flow direction) and block the renal arteries 30, 30′, as shown by the dashed outline 28′ of the upper end 28 of the stent graft shown in FIG. 1. Alternatively, the upper end of the stent graft may become compressed or crumpled, leading to graft/blood vessel seal failure at the entry end 28 of the stent graft 10, allowing fresh blood to enter the excluded aneurysmal sac 16, which may lead to aneurysm growth and eventual rupture.

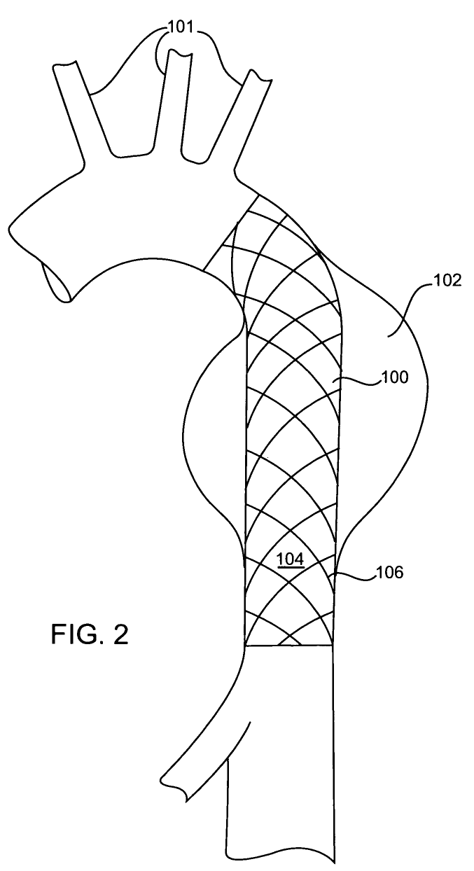

Referring now to FIG. 2, there is shown a stent graft 100, which is located to span a thoracic aneurysm 102. In this embodiment, stent graft 100 must be positioned such that blood flow to the branch arteries 101 in the top of the aortic arch is not blocked. Stent graft 100, like stent graft 10, includes a graft portion 104 and a stent portion 106, the graft portion providing a barrier to blood flow to the aneurysmal sac 108 of the thoracic aneurysm 102, and stent portion 106 providing support thereof to form the graft in a hollow tubular form and cause sealing thereof against the thoracic wall.

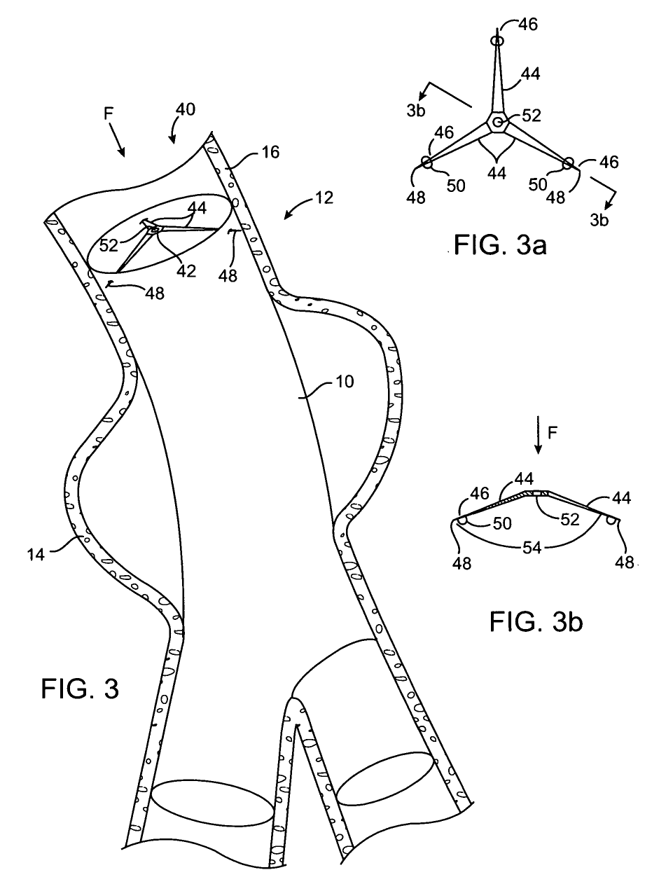

Referring now to FIGS. 3, 3a and 3b, there is shown the placement of the stent graft 10 in an aorta 12 to span an aneurysmal portion 14 therein and a securement device 40 deployed therewith to secure the stent graft, at the blood entry end 28 thereof to the aortal wall 16. Securement device 40, in this embodiment, includes a hub 42, from which a plurality, in this embodiment three, legs 44 extend in an equally spaced relationship about hub 40. Each of the legs 44 terminate in a spike portion 46, having a sharp tip 48 and a stop element 50 deployed thereon inwardly of the tip 48 of the spike portion 46. As also shown in FIG. 3, the tip 48 of each of the spike portion 46 extends through the graft portion 20 of stent graft 10 and through, or alternatively into, aorta wall 16. Additionally, hub 44 includes an aperture 52 extending therethrough, preferably at the center of the hub 42. Additionally, the angle 54 prescribed between adjacent legs is preferably less than 180 degrees. Thus, when properly deployed, the hub 42 of the securement device 40 is positioned slightly upstream, from a blood flow perspective, of the location of the spike portions 46 as they engage the stent graft 10. Thus, blood flowing against the securement device directs force, as shown by arrow F in FIGS. 3 and 3b, in a direction to increase the loading of the spike portions 46 against the stent graft 10, and thereby further increase the ability of the securement device 40 to be maintained in position with the stent graft 10 without the need to otherwise secure the securement device to the stent graft 10, such as by sewing, adhesives, etc. This enables separate delivery of the securement device 40 to the aneurysmal location, as well as relatively simple deployment thereof.

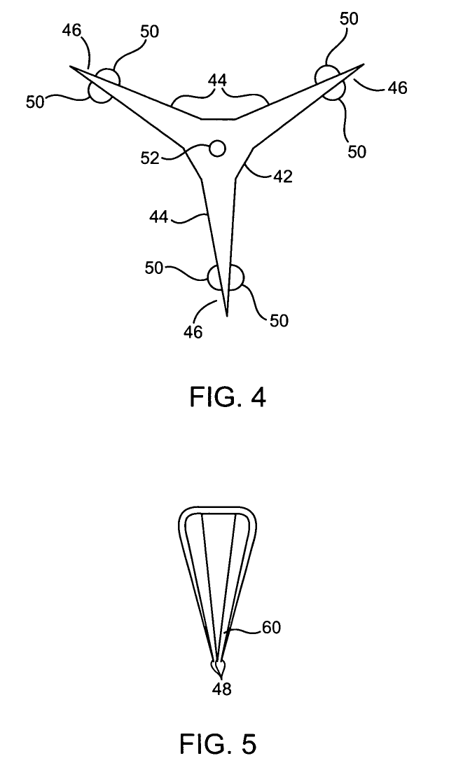

Referring to FIG. 4, there is shown in schematic form the sequence of operations to form the securement device 40. Beginning, with a sheet of shape memory material, such as nitinol, a generally star shaped perform 56 is cut, punched, or otherwise formed therefrom, having a hub 42 with an aperture 52 therethrough, and a plurality, in this embodiment three, legs 44 extending from hub 44 and evenly spaced, at approximately 120 degree separation, from one another, about the periphery of hub 42. Legs 44, when formed, preferably integrally include spike portion 46 and stop elements 50 integrally formed therewith. The preform, when cut, etc., from the sheet of material, will be generally planar, i.e., the hub 42 and each of legs 44 lie in the same plane. Thus, to provide the shape of the securement device 40 as shown in FIG. 3, each of the legs 44 is bent, with respect to the hub 42, at the immediate location of the extension of the legs 44 from the hub, and each leg 44 is bent in the same direction to the same extent, to provide the structure of the preform as shown in FIG. 3. This configuration and alignment of the legs 44 to the hub 42 is selected to ensure that when the securement device of this same configuration is deployed, the ends of the three legs, i.e., the tips 48 of each of the legs 44 contact the inner wall of the stent graft 10 such that a slight tugging of the securement device in a blood flow direction, or downstream of the deployment location, will cause the tips 48 to pierce the graft portion 24 of the stent graft and further extend into the blood vessel, or aorta, wall 16, as will be further described herein.

Once the preform 56 is shaped to the desired securement device shape, the preform is cooled in liquid nitrogen to a temperature on the order of minus 196 degrees Celsius, and the preform is further bent, such as by continuing to bend the legs about the location of their extension from the hub 42, such that an elongated shape having the hub 42 forming one end thereof and the bringing together of the three tips 48 forms the other end thereof, as shown in FIG. 5. A gap 60 remains between the adjacent, closely spaced tips 48. The preform is now in a sufficiently collapsed state such that it may fit into a catheter or tube for intraluminal delivery to an aneurysmal site. It should be appreciated that the preform, when heated back to room temperature or a temperature sufficiently above that of liquid nitrogen, will regain the shape shown in FIG. 4C. The wall of the tube within which the preform is placed prevents this reformation of shape until the securement device 40 is deployed therefrom.

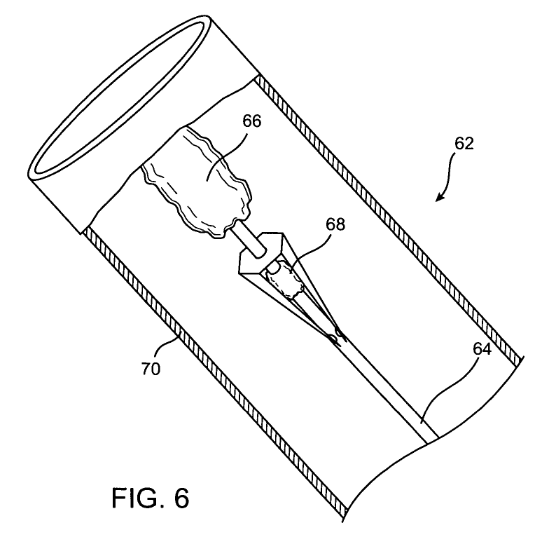

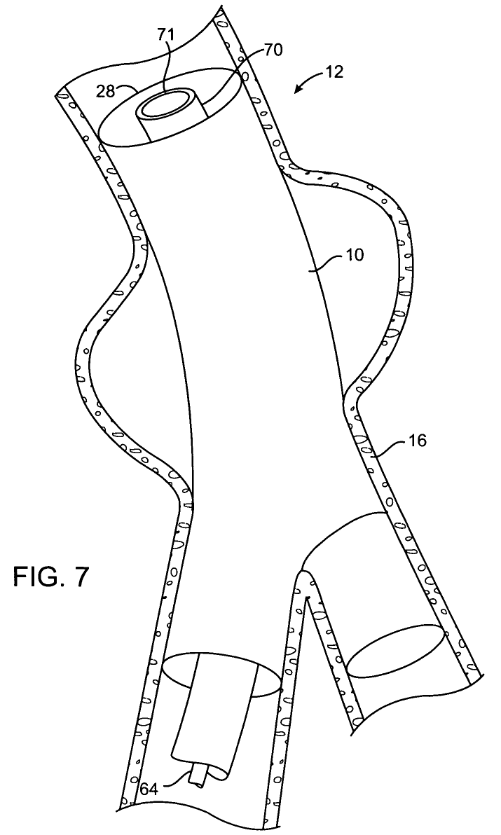

Referring now to FIG. 6, the deployment vehicle 62 for delivering the securement device 40 to the deployment location, and for deployment of the securement device, is shown. Specifically, deployment vehicle 62 includes a hollow push rod 64, extending through the aperture 52 in hub 42. Push rod 64 terminates, adjacent the hub 42, and has an inflatable balloon 66 of the type used for balloon catheterization thereon in a deflated state. Additionally, a second balloon 68 may be provided on push rod 64 and positioned within the envelope of the gap 60 of the folded legs 44 of the securement device 40, and fed from a second fluid channel in the push rod 64. Push rod 64 also includes, on its outer surface thereof intermediate of the two balloon locations, a raised ridge (not shown) of greater diameter than aperture 52 such that push rod 64 engages against the hub 42 when pushed in a first direction, but is free to move within aperture 52 in the opposed direction. Securement device 40, along with balloons 66, 68 and push rod 64, are provided in an intraluminal catheter or delivery tube 70, which is sufficiently long to be inserted in a leg artery and fed up the artery to be located at the aneurysmal location of an aorta or other blood vessel. Likewise, push rod 64 is sufficiently longer than delivery tube 70, such that the end of push rod 64 may be manipulated, with respect to delivery tube 70 by the hand of a surgeon or operator, and holes for providing fluids under pressure to the separate feed conduits of push rod 64 are accessible.

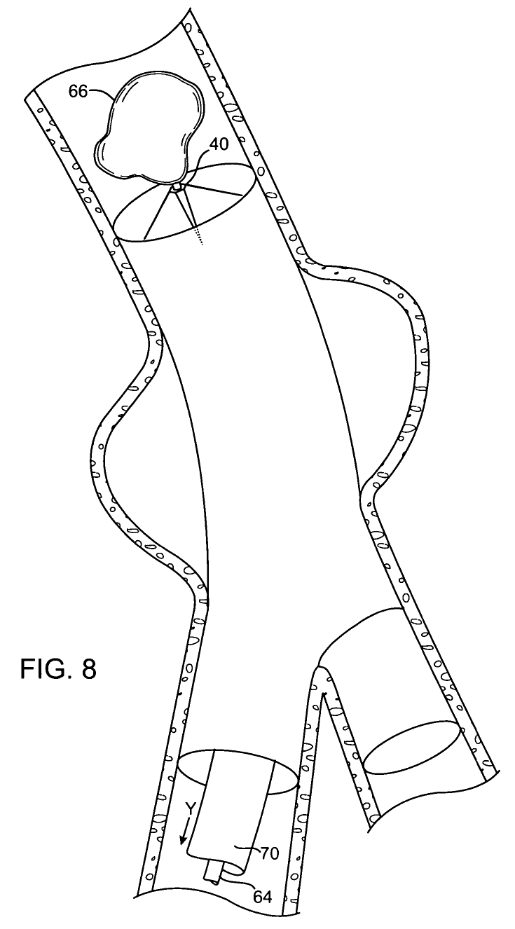

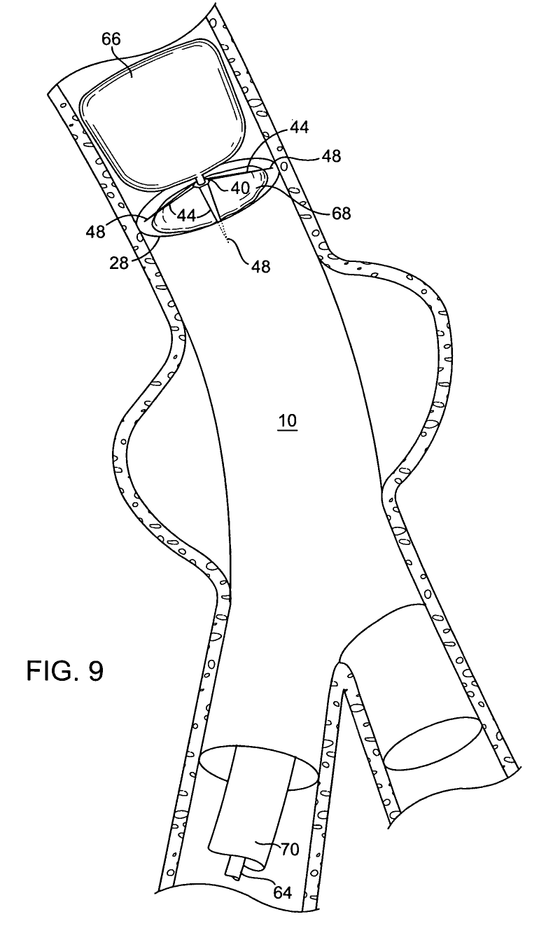

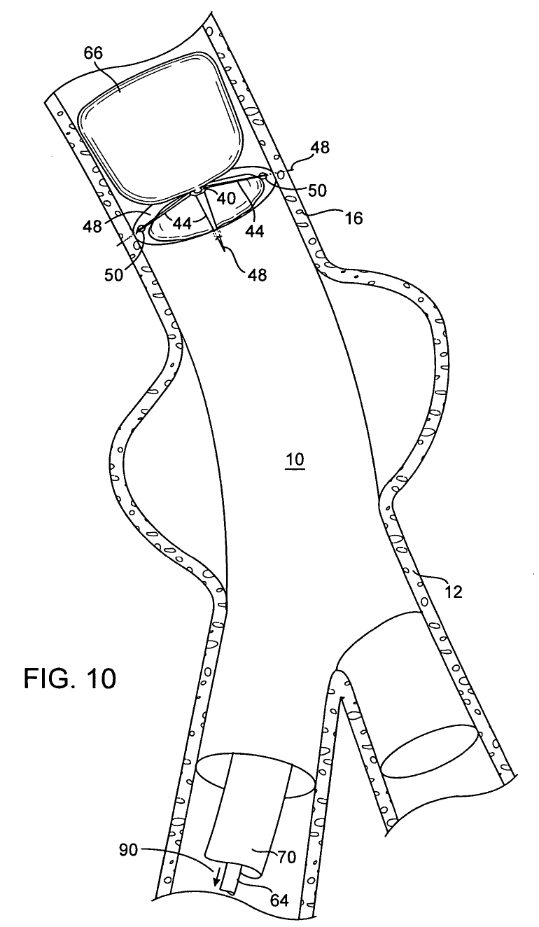

Referring now to FIGS. 7 to 12, there is shown a paradigm for deployment of the stent graft securement device as shown in FIG. 3. In this embodiment, the delivery tube 70 is entered into an incision (not shown) in the leg of a patient, and thence through an incision in the artery therein (not shown), and the end 71 of delivery tube 70 received within the artery, having the securement device 40 therein (not shown in FIG. 7), is pushed up the artery until it is positioned adjacent blood entry end 28 of stent graft 10 as shown in FIG. 7. The position of the stent graft 10 in the aorta 12, as well as the position of the end 71 of the delivery tube 70, may be readily determined by the presence of radiopaque markers (not shown) thereon through the use fluoroscopy, as is well known in the art. Once the delivery tube 70 is in the proper position with respect to blood entry end 28 of the stent graft 10, the surgeon or operator of the delivery device begins withdrawing the delivery tube 70 from the incision thereby pulling the end 71 downwardly through the stent graft 10, in the direction of arrow S in FIG. 8, while holding push rod 64 stationary and inflating first balloon 66, whereby the balloon 66 is now inflated and blocks migration of the securement device off of the tube 70. As delivery tube 70 is further retracted, the tips 480f the legs 44 of the securement device 40 are no longer constrained in motion, and they swing out, as shown in FIG. 8, and continue to swing out until they engage against the graft portion 24 of the stent graft 10 along the inner circumferential (cylindrical) face of the stent graft as shown in FIG. 9. If necessary, such as where the securement device is manufactured of a non-shape memory material, the second balloon 68 is inflated, thereby expanding the lags 44 outwardly a the tips 48 about their intersection with hub 42, to secure tips 48 against the inner face of the graft portion of the stent graft.

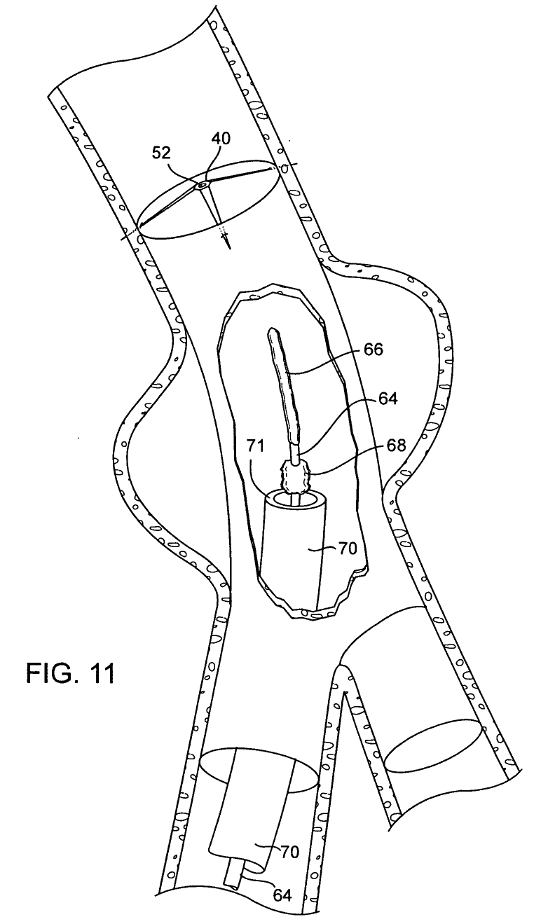

The first balloon 66, provides maintenance of the securement device 40 on the tube 70 during deployment, and also tends to center the tube 70, and thus the securement device 40, within the blood flow entry end 28 of the stent graft 10. Once the securement device 40 is expanded to cause tips 48 thereof the contact the stent graft, then the push rod 64 is moved in a direction to retract it from the incision, causing movement of the securement device 40 in the direction of arrow 90, but only a very small distance sufficient to cause the tips 48 to piece the graft portion 24 of stent graft 10 and aorta wall 16, thereby securing the stent graft 10 in place within the aorta 12 as shown in FIG. 10. The stops 50 on legs 44 prevent excessive penetration of the tips 48 into or through the aorta wall 16, as they bear against the inner surface of the graft portion 24, thus defining the total penetration depth of the tips into the graft and aorta wall 16 as the distance from stop 50 lower or outermost surface to the end of the tip 48. Thus, damage to adjacent organs, which could otherwise occur if tips penetrated them, can be prevented.

Once the securement device is deployed and tips 48 are in securing engagement through the stent graft 10 and in or through aorta wall 16, the push rod 64, as well as delivery tube 70, need be removed from the blood vessel and aorta 12. As shown in FIG. 11, balloons 66, 68 are deflated, such that the push rod 64 may now be pulled through the aperture 52, being prevented previously from doing so by the presence of balloon 66 on the distal end of push rod 64. Thus, as is shown in FIG. 11, the tube 70 and balloon 66 are pulled through aperture 52, leaving securement device 40 anchored in place. Push rod 64 and delivery tube 70 are then withdrawn from the artery and the incisions are sutured shut.

Although the deployment of the securement device 40 has been discussed herein in detail in terms of securing an excluding device, such as a stent graft, located at an ascending aorta 12 location, it is likewise applicable to securing a stent graft at a thoracic aneurysm site. Further, although the securement device had been described herein in terms of specific configurations, and as deployed separately from the deployment of the stent graft, the securement device structure may be modified, and the securement device may be deployed in conjunction with stent graft deployment. Additionally, although the securement device has been described herein in terms of securing a specific device, specifically an excluding device such as a stent graft, it may equally be useful to deploy pharmaceutical type release agents, monitoring devices, or other device for which it would be useful to be secured in a blood vessel location.

The foregoing embodiments of the invention provide anchoring of an intraluminal device with minimal invasiveness to the patient, and with the capability to extend in the flow region of the flow lumen/blood vessel to use the force created by blood flow to further anchor the securement device, while enabling substantial blood flow therethrough. The securement device 40 may be provided during stent graft deployment, the mechanisms of stent graft deployment being well known in the art.

While the foregoing is directed to embodiments of the present invention, other and further embodiments of the invention may be devised without departing from the basic scope thereof.

1. A body flow lumen deployable member, comprising:
at least one spike extendable from the body lumen wall and into a region of fluid flow through a body lumen.

2. The body flow lumen deployable member of claim 1, further including:
a hub receivable in the body flow lumen, said spike connected with said hub.

3. The body flow lumen deployable member of claim 2, further including an arm, extending between and forming the interconnection of said spike and said hub.

4. The body flow lumen deployable member of claim 3, further including a plurality of arms extending from said hub, each of said arms terminating in a spike.

5. The body flow lumen deployable member of claim 1, wherein said body lumen includes an exclusion device therein; and
said spike extends through a portion of said exclusion device and into said flow lumen.

6. The body flow lumen deployable device of claim 5, further including:
a hub;
an arm;
said arm interconnecting said spike and said hub, wherein said spike extending through said exclusion device secures said exclusion device against movement in said body flow lumen.

7. The body flow lumen deployable device of claim 6, further including a plurality of arms extendable from said hub.

8. The body flow lumen deployable device of claim 7, wherein:
each of said arms includes a spike thereon, said spikes each extending through said exclusion device and into said lumen wall.

9. The body flow lumen deployable device of claim 8, wherein said exclusion device is a stent graft.

10. A method of securing an intraluminal device in location in a body flow lumen, comprising:
providing a securement device having a hub portion and at least one arm extending therefrom;
positioning the securement device on a guide rod and positioning the securement device, with the guide rod, at a desired location in a body flow lumen;
inserting, with the rod, a portion of the arm at least into the wall of the body flow lumen;
releasing the securement device from the rod; and
removing the rod from the flow lumen.

11. The method of securing an intraluminal device in location in a body flow lumen of claim 10, further including the steps of:
providing an aperture through the hub portion;
extending the rod through the aperture, the rod bearing against the hub on a first side and extending therefrom on a second side thereof;
providing a hollow region through the rod communicable between a first end of the rod and a second end of the rod; and
positioning an inflatable member on the second end of the rod, the second end of the rod being extendible through the aperture in the hub.

12. The method of securing an intraluminal device in location in a body flow lumen; further including:
inflating the inflatable member when the securement device is located in a flow lumen in a desired position for securement thereof to the flow lumen;
moving the rod in a direction to push the end of the arm inwardly of the flow lumen;
deflating the inflatable member; and
retracting the rod from the flow lumen.

13. The method of securing an intraluminal device in location in a body flow lumen, wherein:
the securement device includes a plurality of arms extending from the hub, each arm generally equally spaced from the next adjacent arm by an equal distance around the perimeter of the hub.

14. The method of securing an intraluminal device in location in a body flow lumen, further including the steps of:
positioning the securement device adjacent to the blood entry location of a lumen exclusion device; and
before extending the portion of the arm inwardly of the flow lumen wall, first extending the arm through the wall of the exclusion device.

15. An intraluminal deployable device, comprising:
a hub
a plurality of arms extending outwardly from said hub;
a tip on each arm;
a depth-limiting member disposed adjacent to said tip;
said arms having a first position, wherein said arms may be brought together to form a small gap between the tips thereof and a second position, wherein said arms are extended outwardly from said hub and engageable with a flow lumen wall.

16. The device of claim 15, wherein said hub and arms are integrally formed of a shape memory material.

17. The device of claim 16, wherein the shape memory material is nitinol.

18. The device of claim 16, further including;
a tube, said tube receiving said hub and said arms therein when said arms are in said folded position;
an aperture in said hub; and
a rod extendible within said tube and at least partially extendible within said aperture.

19. The device of claim 18, further including an inflatable device positioned on the end of said rod extending through said aperture.

20. The device of claim 19, wherein said tube and said rod are slideable with respect to one another.
